(19) United States
(12) Patent Application Publication
(10) Pub. No.: US 2024/0382553 A1
(43) Pub. Date: Nov. 21, 2024

(54) COMPOSITIONS USEFUL FOR IMPROVING THE APPEARANCE OF SKIN AND METHODS OF USE THEREOF

(71) Applicant: Nutraceutical Wellness, Inc, New York, NY (US)

(72) Inventors: Giorgio DELL'ACQUA, New York, NY (US); Michael Weiser, Erie, CO (US); Aleksander Richards, Mount Arlington, NJ (US); Nicole Townsend, New York, NY (US); Ryan McLendon, New York, NY (US)

(73) Assignee: Nutraceutical Wellness, Inc., New York, NY (US)

(21) Appl. No.: 18/663,131

(22) Filed: May 14, 2024

Related U.S. Application Data
(60) Provisional application No. 63/503,454, filed on May 19, 2023.
(60) Provisional application No. 63/584,492, filed on Sep. 21, 2023.

(51) Int. Cl. A61K 36/752; A61K 31/015; A61K 31/22; A61K 31/375; A61K 31/519; A61K 31/593; A61K 33/30; A61K 35/742; A61K 35/747; A61K 36/31; A61K 36/53; A61K 36/63; A61K 36/71; A61K 36/81; A61K 36/888; A61K 36/9066; A61K 36/9068; A61P 17/10

(52) U.S. Cl. CPC A61K 36/752; A61K 31/015; A61K 31/22; A61K 31/375; A61K 31/519; A61K 31/593; A61K 33/30; A61K 35/742; A61K 35/747; A61K 36/31; A61K 36/53; A61K 36/63; A61K 36/71; A61K 36/81; A61K 36/888; A61K 36/9066; A61K 36/9068; A61P 17/10

(57) ABSTRACT

This disclosure relates to skin healthy compositions and methods of use thereof.

### FIELD OF THE INVENTION

This disclosure relates to compositions useful for improving the appearance of skin and methods of use thereof.

### BACKGROUND OF THE INVENTION

Improving skin appearance is highly desirable, with blemishes occurring in children, during adolescence, and persisting into adulthood. Conditions adversely affecting the appearance of skin can arise from many different factors, such as inter alia genetic, hormonal, and environmental factors. Safely and effectively addressing this issue remains very important with compositions and methods for improving the appearance of a human subject's skin remaining a currently unmet need.

### SUMMARY OF THE INVENTION

Provided herein are compositions comprising:
about 1.0% w/w-about 20% w/w of Lepidium meyenii or an extract thereof;
about 1.0% w/w-about 20% w/w of Berberis aristata, an extract thereof or berberine;
about 1.0% w/w-about 25% w/w of Ocimum tenuiflorum or an extract thereof;
about 1.0% w/w-about 20% w/w of Curcuma longa, an extract thereof, or one or more curcuminoids;
about 0.1% w/w-about 10% of Citrus sinensis (L.) Osbeck or an extract thereof;
about 0.1% w/w-about 10% of inactivated Lactobacillus plantarum;
about 0.0001% w/w-about 5.0% w/w of cholecalciferol;
about 0.1% w/w-about 8% w/w of a pharmaceutically acceptable zinc salt or chelate; and
about 0.1% w/w-about 5.0% w/w of a pharmaceutically acceptable selenium salt or chelate.

Also provided herein are methods for improving the appearance of skin of a human subject in need of skin appearance improvement, comprising administering to the human subject an effective amount of the composition described above.

### DETAILED DESCRIPTION OF THE INVENTION

### Definitions

As used herein, the term “about” when immediately preceding a numerical value means±10% of the numerical value.

As used herein, “wt/wt”, “% wt”, “% w/w”, or “% wt/wt” refers to the weight percentage or weight ratio of a particular ingredient to another ingredient or a composition as a whole. In some embodiments, “% wt” refers to the percentage of a particular ingredient compared to the weight of a composition. Unless stated otherwise herein, “wt/wt”, “% wt”, “% w/w”, or “% wt/wt” refers to the weight percentage of a specified ingredient present in a composition. Where a composition is contained in a capsule, “wt/wt”, “% wt”, “% w/w”, or “% wt/wt” excludes the weight of the capsule per se.

As used herein, a “-” symbol between two values means “to”. For example, “about 0.1% w/w-about 5% w/w” means “about 0.1% w/w to about 5% w/w”.

As used herein, “selenium salt or chelate” means selenium salt or selenium chelate.

As used herein, “zinc salt or chelate” means zinc salt or zinc chelate.

### Compositions

Provided herein are compositions useful for improving the appearance of a human subject's skin. Unless otherwise indicated, the amount of an ingredient present in a composition is described using weight for weight mass percentages (“% w/w”).

Without being bound by theory, Lepidium meyenii may help balance hormone levels. In some embodiments, Lepidium meyenii or extract thereof may be present in the compositions provided herein in an amount of about 5.0% w/w-about 15% w/w, such as about 9.5% w/w-about 10.5% w/w.

In some embodiments, Lepidium meyenii or extract thereof may be present in the compositions provided herein in an amount of about 25 mg-about 750 mg, such as about 50 mg.

In some embodiments, the Lepidium meyenii extract is a Lepidium meyenii root extract, a Lepidium meyenii stem extract, a Lepidium meyenii leaf extract, a Lepidium meyenii fruit extract, or a Lepidium meyenii whole plant extract. In some embodiments, the Lepidium meyenii extract is a Lepidium meyenii root extract. In some embodiments, the Lepidium meyenii root extract comprises an extract of dehydrated Lepidium meyenii root. In some embodiments, the Lepidium meyenii root extract comprises an extract of unprocessed Lepidium meyenii root. In some embodiments, the Lepidium meyenii root extract comprises an extract of dehydrated Lepidium meyenii root and an extract of unprocessed Lepidium meyenii root. In some embodiments, the extract of dehydrated Lepidium meyenii root and the extract of unprocessed Lepidium meyenii root are present in the composition at a ratio of about 4:1 w/w, about 3:1 w/w, about 2:1 w/w, about 1:1 w/w, about 4:2 w/w, or about 4:3 w/w.

Without being bound by theory, Berberis aristata may help balance hormone levels. In some embodiments, Berberis aristata or extract thereof or berberine may be present in the compositions provided herein in an amount of about 5% w/w-about 15% w/w, such as about 9.5% w/w-about 10.5% w/w.

In some embodiments, Berberis aristata or extract thereof or berberine may be present in the compositions provided herein in an amount of about 12.5 mg-about 750 mg, such as about 50 mg.

In some embodiments, the Berberis aristata extract is a Berberis aristata root extract, a Berberis aristata stem extract, a Berberis aristata leaf extract, a Berberis aristata fruit extract, or a Berberis aristata whole plant extract. In some embodiments, the Berberis aristata extract is a Berberis aristata root extract. In some embodiments, the Berberis aristata extract is contained in one or more phytosomes. In some embodiments, the one or more phytosomes comprise sunflower lecithin, hydroxypropyl cellulose, pea protein or grape seed extract. In some embodiments, the composition comprises berberine. In some embodiments, the Berberis aristata extract comprises from about 20% to 35% of berberine by weight of the extract, such as about 28% of berberine by weight of the extract.

In some embodiments, Ocimum tenuiflorum or extract thereof may be present in the compositions provided herein in an amount of about 5.0% w/w-about 22.0% w/w. In some embodiments, Ocimum tenuiflorum or extract thereof may be present in the compositions provided herein in an amount of about 6.0% w/w-about 18.0% w/w, such as about 11% w/w- about 13% w/w.

In some embodiments, Ociimum tenuiflorum or extract thereof may be present in the compositions provided herein in an amount of about 25 mg-about 500 mg, such as about 62.5 mg.

In some embodiments, the Ociimum tenuiflorum extract is a Ociimum tenuiflorum root extract, a Ociimum tenuiflorum stem extract, a Ociimum tenuiflorum leaf extract, a Ociimum tenuiflorum fruit extract, or a Ociimum tenuiflorum whole plant extract. In some embodiments, the Ocimum tenuiflorum extract is an Ociimum tenuiflorum leaf extract.

In some embodiments, Curcuma longa or extract thereof or one or more curcuminoids may be present in the compositions provided herein in an amount of about 5.0% w/w-about 15% w/w. In some embodiments, Curcuma longa or extract thereof or one or more curcuminoids may be present in the compositions provided herein in an amount of about 4.5% w/w-about 14.5% w/w, such as about 9.5% w/w-about 10.5% w/w.

In some embodiments, Curcuma longa or extract thereof or one or more curcuminoids may be present in the compositions provided herein in an amount of about 12.5 mg-about 500 mg, such as about 50 mg.

In some embodiments, the Curcuma longa extract is a Curcuma longa root extract, a Curcuma longa stem extract, a Curcuma longa leaf extract, a Curcuma longa fruit extract, or a Curcuma longa whole plant extract. In some embodiments, the Curcuma longa extract is a Curcuma longa rhizome extract.

In some embodiments, the curcumin extract comprises no less than about 20%, no less than about 25%, no less than about 30%, no less than about 40%, no less than about 45%, no less than about 50%, no less than about 55%, or no less than about 60% of one or more curcuminoids by weight of the extract. In some embodiments, the curcumin extract comprises no less than about 45% of one or more curcuminoids by weight of the extract. In some embodiments, the curcumin extract is contained in one or more liposomes.

In some embodiments, Citrus sinensis (L.) Osbeck or extract thereof may be present in the compositions provided herein in an amount of about 0.1% w/w-about 10% w/w. In some embodiments, Citrus sinensis (L.) Osbeck may be present in the compositions provided herein in an amount of about 1.0% w/w-about 10% w/w, such as about 2.0% w/w-about 7.5% w/w.

In some embodiments, Citrus sinensis (L.) Osbeck or extract thereof may be present in the compositions provided herein in an amount of about 1.25 mg-about 250 mg, such as about 25 mg.

In some embodiments, the Citrus sinensis (L.) Osbeck extract is a Citrus sinensis (L.) Osbeck root extract, Citrus sinensis (L.) Osbeck stem extract, a Citrus sinensis (L.) Osbeck leaf extract, a Citrus sinensis (L.) Osbeck fruit extract, or a Citrus sinensis (L.) Osbeck whole plant extract. In some embodiments, the Citrus sinensis (L.) Osbeck extract is a Citrus sinensis (L.) Osbeck fruit extract.

In some embodiments, inactivated Lactobacillus plantarum may be present in the compositions provided herein in an amount of about 1.0% w/w-about 3.7% w/w, such as about 2.2% w/w—about 2.8% w/w.

In some embodiments, inactivated Lactobacillus plantarum may be present in the compositions provided herein in an amount of about 0.25 mg-about 250 mg, such as about 12.5 mg.

In some embodiments, the inactivated Lactobacillus plantarum is heat-killed Lactobacillus plantarum. In some embodiments, the inactivated Lactobacillus plantarum is Lactobacillus plantarum strain L-137.

In some embodiments, cholecalciferol may be present in the compositions provided herein in an amount of about 0.0001% w/w-about 0.005% w/w, such as about 0.001% w/w-about 0.003% w/w. In some embodiments, cholecalciferol may be present in the compositions provided herein in an amount of about 0.40% w/w-about 1.20% w/w, such as about 0.75% w/w-about 0.85% w/w.

In some embodiments, cholecalciferol may be present in the compositions provided herein in an amount of about 1.25 mcg-about 50 mcg, such as about 7.5 mcg to about 12.5 mcg.

In some embodiments, the compositions comprise a pharmaceutically acceptable salt or chelate that is equivalent to about 1.5% w/w-about 5.5% w/w of elemental zinc, such as about 3.5% w/w-about 4.5% w/w of elemental zinc.

In some embodiments, the compositions comprise a pharmaceutically acceptable salt or chelate that is equivalent to about 0.25 mg-about 12.5 mg of elemental zinc, such as about 3.5 mg-about 4 mg of elemental zinc.

In some embodiments, the compositions comprise a pharmaceutically acceptable selenium salt or chelate that is equivalent to about 0.25% w/w-about 0.75% of elemental selenium, such as about 0.50% w/w-about 0.60% w/of elemental selenium.

In some embodiments, the compositions comprise an amount of a pharmaceutically acceptable selenium salt or chelate that is equivalent to about 1.25 mcg-125 mcg of elemental selenium, such as about 40 mcg-about 75 mcg of elemental selenium.

In some embodiments, the compositions provided herein comprise Olea europaea (olive) or an extract thereof. In some embodiments, Olea europaea may be present in the compositions provided herein in an amount of about 0.5% w/w-about 10% w/w, such as about 0.75% w/w-about 2.25% w/w.

In some embodiments, Olea europaea may be present in the compositions provided herein in an amount of about 1.25 mg-about 250 mg, such as about 5 mg-about 10 mg.

In some embodiments, the Olea europaea extract is an Olea europaea root extract, an Olea europaea fruit extract, an Olea europaea leaf extract, or an Olea europaea whole plant extract. In some embodiments, the Olea europaea extract is an Olea europaea fruit extract. In some embodiments, the Olea europaea extract comprises about 5%, about 10%, about 15%, about 20%, about 25%, about 30%, about 35%, about 40%, about 45%, or about 50% hydroxytyrosol by weight of the Olea europaea extract. In some embodiments, the Olea europaea extract comprises about 20% hydroxytyrosol by weight of the Olea europaea extract.

In some embodiments, the compositions provided herein comprise Amorphophallus konjac (konjac) or an extract thereof. In some embodiments, Amorphophallus konjac may be present in the compositions provided herein in an amount of about 1% w/w-about 15% w/w, such as about 4.25% w/w-about 5.75% w/w.

In some embodiments, Amorphophallus konjac may be present in the compositions provided herein in an amount of about 2.5 mg-about 375 mg, such as about 15 mg-about 50 mg.

In some embodiments, the Amorphophallus konjac extract is a Amorphophallus konjac root extract, a Amorphophallus konjac fruit extract, a Amorphophallus konjac leaf extract, or a Amorphophallus konjac whole plant extract. In some embodiments, the Amorphophallus konjac extract is a Amorphophallus konjac root extract. In some embodiments, the Amorphophallus konjac extract comprises no less than about 1%, no less than about 2%, no less than about 3%, no less than about 4%, no less than about 5%, no less than about 6%, no less than about 7%, no less than about 8%, no less than about 9%, no less than or about 10% of one or more ceramides by weight of the Amorphophallus konjac extract. In some embodiments, the Amorphophallus konjac extract comprises no less than about 5% of one or more ceramides by weight of the Amorphophallus konjac extract.

In some embodiments, the compositions provided herein comprise Solanum lycopersicum (tomato) or an extract thereof. In some embodiments, Solanum lycopersicum may be present in the compositions provided herein in an amount of about 1% w/w-about 10% w/w. In some embodiments, Solanum lycopersicum may be present in the compositions provided herein in an amount of about 1.75% w/w-about 5.25% w/w.

In some embodiments, Solanum lycopersicum may be present in the compositions provided herein in an amount of about 1.25 mg-about 250 mg, such as about 15 mg-about 20 mg.

In some embodiments, the Solanum lycopersicum extract is a Solanum lycopersicum root extract, a Solanum lycopersicum fruit extract, a Solanum lycopersicum leaf extract, or a Solanum lycopersicum whole plant extract. In some embodiments, the Solanum lycopersicum extract is a Solanum lycopersicum fruit extract. In some embodiments, the Solanum lycopersicum extract comprises no less than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of lycopene by weight of the Solanum lycopersicum extract. In some embodiments, the Solanum lycopersicum extract comprises no less than about 10% of lycopene by weight of the Solanum lycopersicum extract.

In some embodiments, the compositions provided herein comprise Zingiber officinale (ginger) or an extract thereof. In some embodiments, Zingiber officinale may be present in the compositions provided herein in an amount of about 0.1% w/w-about 5% w/w, such as about 0.65% w/w-about 0.85% w/w.

In some embodiments, Zingiber officinale may be present in the compositions provided herein in an amount of about 1.25 mg-about 750 mg, such as about 2.5 mg-about 5 mg.

In some embodiments, the Zingiber officinale extract is a Zingiber officinale root extract, a Zingiber officinale stem extract, a Zingiber officinale leaf extract, a Zingiber officinale fruit extract, a Zingiber officinale rhizome extract, or a Zingiber officinale whole plant extract. In some embodiments, the Zingiber officinale extract is a Zingiber officinale rhizome extract. In some embodiments, the Zingiber officinale extract comprises no less than about 1%, no less than about 2%, no less than about 3%, no less than about 4%, no less than about 5%, no less than about 6%, no less than about 7%, no less than about 8%, no less than about 9%, or no less than about 10% of one or more gingerols by weight of the Zingiber officinale extract. In some embodiments, the Zingiber officinale extract comprises no less than about 5% of one or more gingerols by weight of the Zingiber officinale extract.

In some embodiments, the compositions provided herein comprise tributyrin. In some embodiments, tributyrin may be present in the compositions provided herein in an amount of about 1% w/w-about 20% w/w, such as about 7.75% w/w-about 11.75% w/w.

In some embodiments, tributyrin may be present in the compositions provided herein in an amount of about 2.5 mg-about 375 mg, e.g., about 25 mg-about 75 mg

In some embodiments, the compositions provided herein comprise Bacillus subtilis DE111. In some embodiments, Bacillus subtilis DE111 may be present in the compositions provided herein in an amount of about 0.1% w/w-about 5% w/w, such as about 0.45% w/w-about 0.75% w/w.

In some embodiments, Bacillus subtilis DE111 may be present in the compositions provided herein in an amount of about 0.25 mg-about 50 mg, such as 1.25 mg-about 5 mg.

In some embodiments, about 10 mg of Bacillus subtilis DE111 comprises about one billion colony forming units.

In some embodiments, the compositions provided herein comprise beta-carotene. In some embodiments, beta-carotene may be present in the compositions provided herein in an amount of about 0.01% w/w-about 5% w/w, such as about 0.05% w/w-about 0.95% w/w.

In some embodiments, beta-carotene may be present in the compositions provided herein in an amount of about 1.25 mcg-about 10 mg, such as about 0.25 mcg-about 5 mcg.

In some embodiments, the compositions provided herein comprise vitamin C. In some embodiments, vitamin C may be present in the compositions provided herein in an amount of about 0.25% w/w-about 10% w/w, such as about 1.0% w/w-about 3.4% w/w.

In some embodiments, vitamin C may be present in the compositions provided herein in an amount of about 1.25 mg-about 500 mg, such as about 7.5 mg to about 12.5 mg.

In some embodiments, the compositions provided herein comprise a form of vitamin B3 such as niacinamide. In some embodiments, vitamin B3 may be present in the compositions provided herein in an amount of about 1.0% w/w-about 20.0% w/w, such as about 8.0% w/w-about 10.0% w/w.

In some embodiments, vitamin B3 may be present in the compositions provided herein in an amount of about 1.25 mg-about 375 mg, such as about 25 mg-about 50 mg.

In some embodiments, the compositions provided herein comprise a form of vitamin B5 such as d-calcium pantothenate. In some embodiments, vitamin B5 may be present in the compositions provided herein in an amount of about 0.1% w/w-about 10% w/w, such as about 1.2% w/w-about 1.8% w/w.

In some embodiments, vitamin B5 may be present in the compositions provided herein in an amount of about 0.25 mg-about 125 mg, such as about 2.5 mg to about 7.5 mg.

In some embodiments, the compositions provided herein comprise L-5-methyltetrahydrofolate or a pharmaceutically acceptable salt thereof., such as calcium L-5-methyltetrahydrofolate. In some embodiments, L-5-methyltetrahydrofolate or a pharmaceutically acceptable salt thereof may be present in the compositions provided herein in an amount that is equivalent to about 0.001% w/w-about 1% w/w of L-5-methyltetrahydrofolate, such as about 0.025% w/w-about 0.035% w/w.

In some embodiments, L-5-methyltetrahydrofolate or a pharmaceutically acceptable salt thereof may be present in the compositions provided herein in an amount that is equivalent to about 2.5mcg-about 250 mcg of L-5-methyltetrahydrofolate, such as about 75 mcg-about 175 mcg.

In some embodiments, the compositions described herein comprise about 25 mg-about 750 mg of Lepidium meyenii or an extract thereof; about 12.5 mg-about 750 mg of Berberis aristata, an extract thereof or berberine; about 25 mg-about 500 mg of Ociimum tenuiflorum or an extract thereof; about 12.5 mg-about 500 mg of Curcuma longa, an extract thereof or one or more curcuminoids; about 1.25 mg-about 250 mg of Citrus sinensis (L.) Osbeck or an extract thereof; about 0.25 mg-about 250 mg of inactivated Lactobacillus plantarum; about 1.25 mcg-about 50 mcg of cholecalciferol; an amount of a pharmaceutically acceptable zinc salt or chelate that is equivalent to about 0.25 mg-about 12.5 mg of elemental zinc; and an amount of a pharmaceutically acceptable selenium salt or chelate that is equivalent to about 1.25 mcg-about 125 mcg of elemental selenium.

In some embodiments, the compositions described herein comprise about 4.5% w/w-about 14.5% w/w of Lepidium meyenii or an extract thereof; about 4.5% w/w-about 14.5% w/w of Berberis aristata, an extract thereof or berberine; about 6.0% w/w-about 18.0% w/w of

Ociimum tenuiflorum or an extract thereof; about 4.5% w/w-about 14.5% w/w of Curcuma longa, an extract thereof or one or more curcuminoids; about 2.0% w/w-about 7.5% w/w of Citrus sinensis (L.) Osbeck or an extract thereof; about 1.0% w/w-about 3.7% w/w of inactivated Lactobacillus plantarum; about 0.0001% w/w-about 1.20% w/w of cholecalciferol; about 1.5% w/w-about 5.5% of a pharmaceutically acceptable zinc salt or chelate; and about 0.25% w/w-about 0.75% of a pharmaceutically acceptable selenium salt or chelate.

In some embodiments, the compositions described herein further comprise about 1.25 mg-about 250 mg of Olea europaea or an extract thereof, about 2.5 mg-about 375 mg of Amorphophallus konjac or an extract thereof, about 1.25 mg-about 250 mg of Solanum lycopersicum or an extract thereof, about 1.25 mg-about 750 mg of Zingiber officinale or an extract thereof, about 2.5 mg-about 375 mg of tributyrin, about 0.25 mg-about 50 mg of Bacillus subtilis DE111, about 1.25 mcg-about 10 mg of beta-carotene, about 1.25 mg-about 500 mg of vitamin C, about 1.25 mg-about 375 mg of vitamin B3, about 0.25 mg-about 125mg of vitamin B5 or an amount of a pharmaceutically acceptable salt of vitamin B5 that is equivalent to about 0.25 mg-125 mg of vitamin B5, or about 2.5 mcg-about 250 mcg of L-5-methyltetrahydrofolate or an amount of a pharmaceutically acceptable salt of L-5- methyltetrahydrofolate that is equivalent to about 2.5 mcg-about 250 mcg of L-5-methyltetrahydrofolate.

In some embodiments, the compositions described herein further comprise about 0.75% w/w-about 2.25% w/w of Olea europaea or an extract thereof, about 2.25% w/w-about 7.25% w/w of Amorphophallus konjac or an extract thereof, about 1.75% w/w-about 5.25% w/w of Solanum lycopersicum or an extract thereof, about 0.25% w/w-about 1.25% w/w of Zingiber officinale or an extract thereof, about 4.75% w/w-about 14.75% w/w of tributyrin, about 0.25% w/w-about 0.95% w/w of Bacillus subtilis DE111, about 0.05% w/w-about 0.95% w/w of beta-carotene, about 1.0% w/w-about 3.4% w/w of vitamin C, about 4.0% w/w-about 13.0% w/w of vitamin B3, about 0.6% w/w-about 2.2% w/w of vitamin B5 or an amount of a pharmaceutically acceptable salt of vitamin B5 that is equivalent to about 0.6% w/w-about 2.2% w/w of vitamin B5, or about 0.015% w/w-about 0.045% w/w of L-5-methyltetrahydrofolate or an amount of a pharmaceutically acceptable salt of L-5-methyltetrahydrofolate that is equivalent to about 0.015% w/w of L-5-methyltetrahydrofolate- about 0.045% w/w of L-5-methyltetrahydrofolate.

In some embodiments, the compositions are formulated for oral administration, e.g., contained in a capsule, as a tablet, as a beverage or as a gummy, lozenge, powder, confectionary or food.

In some embodiments, the compositions are contained in a capsule. In some embodiments, the compositions are in the form of a tablet, e.g., a chewable tablet. In some embodiments, the compositions are in the form of a beverage, e.g., a ready-to-drink beverage or a beverage shot.

In some embodiments, the compositions are unit dosage forms comprising about 100 mg to about 1500 mg of the compositions described herein, such as about 500 mg to about 600 mg of the compositions described herein.

In some embodiments, the compositions are contained in capsules of size 000, 00, 0, 1, 2, 3 or 4.

### Excipients and Carriers

In some embodiments, the compositions provided herein further comprise a cosmetically acceptable carrier or excipient. In some embodiments, the carrier is an alcohol-free carrier. Alternatively, the carrier or excipient may comprise the balance of the composition after considering the amount of the other components used.

In some embodiments, the compositions may further comprise one or more inactive ingredients, such as one or more surfactants, co-solvents, and excipients or fillers (e.g., solid, semi-solid, liquid, etc.); emollients; delivery enhancers; circulation enhancers; antimicrobial agents; anti-inflammatory agents; foaming agents; carriers; diluents (e.g., organic rice hull concentrate); binding agents (e.g., dextran); thickening agents (e.g., rice maltodextrin); gelling agents;

vitamins, retinoids, and retinols (e.g., vitamin B3, beta-carotene, etc.); pigments; fragrances; sunscreens and sunblocks; anti-oxidants and radical scavengers (e.g., tocopheryl acetate or vitamin E acetate); organic hydroxy acids; exfoliants; skin conditioners (e.g., ethylhexylglycerin, hydrolyzed soy protein, glycol distearate, cyclopentasiloxane, quaternium-79 hydrolyzed keratin, propylene glycol, etc.); moisturizers; humectants (e.g., hydrolyzed soy protein, propylene glycol, etc.); ceramides, pseudoceramides; phospholipids, sphingolipids, cholesterol, glucosamine; pharmaceutically acceptable penetrating agents (e.g., n-decylmethyl sulfoxide, lecithin organogels, tyrosine, lysine, etc.); preservatives (e.g., phenoxyethanol, benzoic acid, dehydroacetic acid, polyaminopropyl biguanide, DMDM hydantoin or 1,3-bis(hydroxymethyl)-5,5-dimethylimidazolidine-2,4-dione, iodopropynyl butylcarbamate, iodopropynyl butylcarbonate, stearalkonium chloride, etc.); amino acids such as proline; pyrrolidone carboxylic acid, its derivatives and salts; saccharide isomerate; panthenol (i.e., provitamin of B5); buffers together with a base such as triethanolamine or sodium hydroxide; waxes, such as beeswax, ozokerite wax, paraffin wax; plant extracts, obtained from plants such as Aloe vera leaf, cornflower, witch hazel, elderflower, green tea (e.g., Camellia sinensis) leaf, grape (e.g., Vitis vinifera) seed, jojoba (e.g., Simmondsia chinensis) seed, tea tree (e.g., Malaleuca alternifolia) leaf, rosemary (e.g., Rosmarinus officinalis), henna, sunflower (e.g., Helianthus annuus) seed, wild soybean (e.g., Glycine soja), argan tree kernel or argan, cucumber, shiso, etc.; or combinations thereof. As will be appreciated by one of skill in the art viewing this disclosure, the selection and amount of optional ingredients may be varied.

In some embodiments, one or more inactive ingredients are present in the compositions provided herein in an amount of 1% w/w-about 30% w/w, such as h. In some embodiments, one or more inactive ingredient(s) are present in the compositions provided herein in an amount of 2.0% w/w-about 20.0% w/w, e.g., about 3.0% w/w-about 4.0% w/w, about 4.0% w/w-about 5.0% w/w, about 5.0% w/w-about 6.0% w/w, about 6.0% w/w-about 7.0% w/w, about 7.0% w/w-about 8.0% w/w, about 8.0% w/w-about 9.0%, about 9.0% w/w-about 10.0,about 10.0% w/w-about 11.0%, about 11.0% w/w-about 12.0%, about 12.0% w/w-about 13.0%, about 13.0% w/w-about 14.0%, about 14.0% w/w-about 15.0%, about 15.0% w/w-about 16.0%, about 16.0% w/w-about 17.0%, about 17.0% w/w-about 18.0%, about 18.0% w/w-about 19.0%, about 19.0% w/w-about 20.0%, or about 20.0% w/w-about 30.0%.

In some embodiments, the compositions provided herein further comprise organic rice hull concentrate. In some embodiments, organic rice hull concentrate may be present in the compositions provided herein in an amount of about 1 mg-about 10 mg, about 5 mg to-about 10 mg, about 10 mg-about 70 mg, about 20 mg-about 25 mg, about 25 mg-about 30 mg, about 30 mg-about 35 mg, about 35 mg-about 40 mg, about 40 mg-about 45 mg, about 45 mg-about 50 mg, about 55 mg-about 60 mg, about 60 mg-about 65 mg, or about 65 mg - about 70 mg. In some embodiments, organic rice hull concentrate is present in the compositions in an amount of about 43 mg. In some embodiments, organic rice hull concentrate is present in the compositions in an amount of about 8.2 mg.

In some embodiments, organic rice hull concentrate may be present in the compositions provided herein in an amount of 1% w/w-about 20% w/w. In some embodiments, organic rice hull concentrate may be present in the compositions provided herein in an amount of 0.8% w/w-about 3.5% w/w, e.g., about 0.8% w/w-about 1.0% w/w, about 1.2% w/w-about 1.4% w/w, about 1.4% w/w-about 1.6% w/w, about 1.6% w/w-about 1.8% w/w, about 1.8% w/w-about 2.0% w/w, about 2.0% w/w-about 2.5% w/w, about 2.5% w/w-about 3.0%, or about 3.0% w/w-about 3.5% w/w. In some embodiments, organic rice hull concentrate is present in the compositions in an amount of about 1.7% w/w. In some embodiments, organic rice hull concentrate may be present in the compositions provided herein in an amount of 4.0% w/w-about 12.0% w/w, e.g., about 4.0% w/w-about 5.0% w/w, about 5.0% w/w-about 6.0% w/w, about 6.0% w/w-about 7.0% w/w, about 7.0% w/w-about 8.0% w/w, about 8.0% w/w-about 9.0% w/w, about 9.0% w/w-about 10.0% w/w, about 10.0% w/w-about 11.0% w/w, or about 11.0% w/w-about 12.0% w/w. In some embodiments, organic rice hull concentrate is present in the compositions in an amount of about 8.2% w/w.

In some embodiments, the compositions provided herein further comprise rice maltodextrin. In some embodiments, rice maltodextrin may be present in the compositions provided herein in an amount of about 1 mg-about 30 mg, about 5 mg-about 30 mg, about 15 mg-about 30 mg, about 15 mg-about 50 mg, e.g., about 15 mg-about 20 mg, about 20 mg-about 25 mg, about 25 mg-about 30 mg, about 30 mg-about 35 mg, about 35 mg-about 40 mg, about 40 mg-about 45 mg, or about 45 mg-about 50 mg. In some embodiments, rice maltodextrin is present in the compositions in an amount of about 33 mg. In some embodiments, rice maltodextrin is present in the compositions in an amount of about 23 mg.

In some embodiments, rice maltodextrin may be present in the compositions provided herein in an amount of 1% w/w-about 15% w/w, such as about 4.0% w/w-about 7.0% w/w. In some embodiments, the compositions of the invention further comprise sodium C14-16 olefin sulfonate, cetearyl alcohol, cocamidopropyl betaine, brassicamidopropyl dimethylamine, propanediol, sodium lauroyl sarcosinate, parfum (fragrance), sodium chloride, lactic acid, piroctone olamine, caprylyl/capryl glucoside, guar hydroxypropyltrimonium chloride, diisopropyl adipate, tetrasodium glutamate diacetate, glycol distearate, triethyl citrate, benzyl alcohol, sodium benzoate, potassium sorbate, cocamide MIPA, sodium hydroxide, hydroxypropyl methylcellulose, polyquaternium-37, isopropyl alcohol, citral, geraniol, caprylyl glyceryl ether, limonene, tetrasodium EDTA, linalool, citric acid, or CI 77289 (chromium hydroxide green). In some embodiments, the compositions provided herein comprise one or more of water, glycerin, Camellia sinensis (green tea) leaf or an extract thereof, glycine, larix europaea wood or an extract thereof, sodium metabisulfite, zinc chloride, Pisum sativum (pea) sprout or an extract thereof, alcohol, Olea europaea (olive) leaf or an extract thereof, Curcuma longa (turmeric) root or an extract thereof, Equisetum arvense (horsetail) or an extract thereof, Hippophae rhamnoides (sea buckthorn) fruit oil, Laminaria saccharina (neptune kelp) or an extract thereof, Lepidium meyenii (maca) root or an extract thereof, Melaleuca alternifolia (tea tree) leaf oil, Moringa oleifera (moringa) leaf or an extract thereof, Panax ginseng (ginseng) root or an extract thereof, DL-panthenol, L-theanine, Melatonin, Niacinamide, sodium dehydroacetate, sodium hyaluronate, and phytic acid.

In some embodiments, the compositions described herein do not comprise a paraben. In some embodiments, the compositions described herein do not comprise a silicone. In some embodiments, the compositions described herein do not comprise a phthalate. In some embodiments, the compositions described herein do not comprise a synthetic fragrance. In some embodiments, the compositions described herein do not comprise a synthetic hormone. In some embodiments, the compositions described herein are essentially free of parabens. In some embodiments, the compositions described herein are essentially free of silicones. In some embodiments, the compositions described herein are essentially free of phthalates. In some embodiments, the compositions described herein are essentially free of synthetic fragrances. In some embodiments, the compositions described herein are essentially free of synthetic hormones. “As used herein, “essentially free” of a particular ingredient means that the compositions comprise no more than about 1% w/w of the ingredient.

In some embodiments, the compositions described herein are formulated for oral administration. Thus, in some embodiments, provided herein are capsules, tablets, or other dosage forms comprising the compositions described herein. In some embodiments, provided herein are capsules containing the compositions described herein. In some embodiments, the capsule is a gelatin capsule or a hydroxypropyl methylcellulose capsule.

In some embodiments, the dosage forms comprise about 25 mg-about 5 g of a composition described herein, such as about 450 mg-about 550 mg of the composition.

In some embodiments, a capsule shell has a mass of about 25 mg-about 500 mg, such as a capsule shell has a mass of about 125 mg.

In some embodiments, provided herein are capsules containing a composition comprising or consisting essentially of:
about 1.0% w/w-about 20% w/w of Lepidium meyenii or an extract thereof;
about 1.0% w/w-about 20% w/w of Berberis aristata, an extract thereof or berberine;
about 1.0% w/w-about 25% w/w of Ociimum tenuiflorum or an extract thereof;
about 1.0% w/w-about 20% w/w of Curcuma longa, an extract thereof, or one or more curcuminoids;
about 0.1% w/w-about 10% of Citrus sinensis (L.) Osbeck or an extract thereof;
about 0.1% w/w-about 10% of inactivated Lactobacillus plantarum;
about 0.0001% w/w-about 5.0% w/w of cholecalciferol;
about 0.1% w/w-about 8% w/w of a pharmaceutically acceptable zinc salt or chelate; and
about 0.1% w/w-about 5.0% w/w of a pharmaceutically acceptable selenium salt or chelate.

In some embodiments, provided herein are capsules containing a composition comprising or consisting essentially of:
about 25 mg-about 750 mg of Lepidium meyenii or an extract thereof;
about 12.5 mg-about 750 mg of Berberis aristata, an extract thereof or berberine;
about 25 mg-about 500 mg of Ociimum tenuiflorum or an extract thereof;
about 12.5 mg-about 500 mg of Curcuma longa, an extract thereof or one or more curcuminoids;
about 1.25 mg-about 250 mg of Citrus sinensis (L.) Osbeck or an extract thereof;
about 0.25 mg-about 250 mg of inactivated Lactobacillus plantarum;
about 1.25 mcg-about 50 mcg of cholecalciferol;
an amount of a pharmaceutically acceptable zinc salt or chelate that is equivalent to about 0.25mg-about 12.5 mg of elemental zinc; and
an amount of a pharmaceutically acceptable selenium salt or chelate that is equivalent to about 1.25 mcg-about 125 mcg of elemental selenium.

In some embodiments, provided herein are capsules containing a composition comprising or consisting essentially of:
about 1.0% w/w-about 20% w/w of Lepidium meyenii or an extract thereof;
about 1.0% w/w-about 20% w/w of Berberis aristata, an extract thereof or berberine;
about 1.0% w/w-about 25% w/w of Ociimum tenuiflorum or an extract thereof;
about 1.0% w/w-about 20% w/w of Curcuma longa, an extract thereof, or one or more curcuminoids;
about 0.1% w/w-about 10% of Citrus sinensis (L.) Osbeck or an extract thereof;
about 0.1% w/w-about 10% of inactivated Lactobacillus plantarum;
about 0.0001% w/w-about 5.0% w/w of cholecalciferol;
an amount of a pharmaceutically acceptable zinc salt or chelate that is equivalent to about 0.1% w/w-about 8% w/w of elemental zinc;
an amount of a pharmaceutically acceptable selenium salt or chelate that is equivalent to about 0.1% w/w-about 5.0% w/w of elemental selenium;
about 0.5% w/w-about 10% w/w of Olea europaea or an extract thereof;
about 1% w/w-about 15% w/w of Amorphophallus konjac or an extract thereof;
about 1% w/w-about 10% w/w of Solanum lycopersicum or an extract thereof;
about 0.125% w/w-about 5% w/w of Zingiber officinale or an extract thereof;
about 1% w/w-about 20% w/w of tributyrin;
about 0.1% w/w-about 5% w/w of Bacillus subtilis DE111;
about 0.01% w/w-about 5% w/w of beta-carotene;
about 0.25% w/w-about 10% w/w of vitamin C;
about 1.0% w/w-about 20.0% w/w of vitamin B3;
about 0.1% w/w-about 10% w/w of vitamin B5, or an amount of a pharmaceutically acceptable vitamin B5 salt that is equivalent to about 0.1% w/w-about 10% w/w of vitamin B5; and
about 0.005% w/w-about 1% w/w of L-5-methyltetrahydrofolate, or an amount of a pharmaceutically acceptable L-5-methyltetrahydrofolate salt that is equivalent to about 0.005% w/w-about 1% w/w of L-5-methyltetrahydrofolate.

In some embodiments, provided herein are capsules containing a composition or consisting essentially of:
about 25 mg-about 750 mg of Lepidium meyenii or an extract thereof;
about 12.5 mg-about 750 mg of Berberis aristata, an extract thereof or berberine;
about 25 mg-about 500 mg of Ociimum tenuiflorum or an extract thereof;
about 12.5 mg-about 500 mg of Curcuma longa, an extract thereof or one or more curcuminoids;
about 1.25 mg-about 250 mg of Citrus sinensis (L.) Osbeck or an extract thereof;
about 0.25 mg-about 250 mg of inactivated Lactobacillus plantarum;
about 1.25 mcg-about 50 mcg of cholecalciferol;
an amount of a pharmaceutically acceptable zinc salt or chelate that is equivalent to about 0.25 mg-about 12.5 mg of elemental zinc;
an amount of a pharmaceutically acceptable selenium salt or chelate that is equivalent to about 1.25 mcg-about 125 mcg of elemental selenium;
about 1.25 mg-about 250 mg of Olea europaea extract;
about 2.5 mg-about 375 mg of Amorphophallus konjac extract;
about 1.25 mg-about 250 mg of Solanum lycopersicum extract;
about 3.75 mg of Zingiber officinale extract;
about 2.5 mg-about 375 mg of tributyrin;
about 0.25 mg-about 50 mg of Bacillus subtilis DE111;
about 1.25 mcg-about 10 mg of beta-carotene;
about 1.25 mg-about 500 mg of vitamin C;
about 1.25 mg-about 375 mg of vitamin B3;
about 0.25 mg-about 125 mg of vitamin B5 or an amount of a pharmaceutically acceptable salt of vitamin B5 that is equivalent to about 0.25 mg-125 mg of vitamin B5; and
about 2.5 mcg-about 250 mcg of L-5-methyltetrahydrofolate or an amount of a pharmaceutically acceptable salt of L-5-methyltetrahydrofolate that is equivalent to about 2.5 mcg-about 250 mcg of L-5-methyltetrahydrofolate.

### Methods of Use

Also provided herein are methods useful for improving the appearance of a human subject's skin, comprising administering to a human subject in need thereof an effective amount of a composition described herein. The human subject to whom the compositions and dosage forms described herein are administered may suffer from a skin condition, for example, the human subject may have or be suspected of having, acne, atopic or seborrheic dermatitis, psoriasis, skin hyperpigmentation, sunburn, skin erythema, sunspots, wrinkles, fine lines, poor skin hydration, or general skin inflammation. In embodiments wherein the human subject has acne, the acne may be non-inflammatory or inflammatory acne. In embodiments wherein the human subject has poor skin hydration, the poor skin hydration may be dry, itchy, cracked, or dehydrated skin.

In some embodiments, the human subject is biologically female. In some embodiments, the human subject is an adult.

The compositions described herein may be administered to a human subject using any suitable dose regimen. The compositions described herein may be administered at least once a week, such as at least every two days, or at least once each day. For example, application may be twice per day. In general, administration of the compositions described herein, may be continued indefinitely. Alternatively, the administration may be repeated only for a limited period, e.g., several weeks or months.

The compositions described herein may be advantageously used to improve the appearance of a human subject's skin. For example, as disclosed herein, the compositions described herein may improve the appearance of a human subject's skin in a time period of from about 4 weeks to about 20 weeks, alternatively from about 4 weeks to about 16 weeks, or alternatively from about 4 weeks to about 10 weeks. In some embodiments, the compositions described herein are administered for about 4 weeks, about 8 weeks, about 12 weeks, about 16 weeks, about 20 weeks, about 24 weeks, about 28 weeks, about 32 weeks, about 36 weeks, about 40 weeks, about 48 weeks, or about 52 weeks.

In some embodiments, the compositions described herein are contained within a capsule. In some embodiments, one, two, three, four, five, six, or more capsules or capsules provided herein are administered to the human subject per day. In some embodiments, a daily dose ranges from 1-10 capsules disclosed herein. In some embodiments, the daily dose ranges from 2-6 capsules disclosed herein. In some embodiments, the daily dose is four capsules provided herein.

In some embodiments, a human subject is administered a daily dose of about 25 mg-about 5 g of a composition described herein. In some embodiments, a human subject is administered a daily dose of about 500 mg to about 2,500 mg of the composition. In some embodiments, the daily dose is divided into several capsules. In some embodiments, the daily dose is divided into 2 capsules. In some embodiments, the daily dose is divided into 3 capsules. In some embodiments, the daily dose is divided into 4 capsules. In some embodiments, the daily dose is divided into 5 capsules. In some embodiments, the daily dose is divided into 6 capsules.

Any suitable method known in the art or described herein may be used to assess the efficacy of the methods described herein. In some embodiments, methods for improving the appearance of a human subject's skin described herein result in an improvement of one or more parameters compared to baseline, i.e., compared to the same parameter measurement before the administration begins.

In some embodiments, methods for improving the appearance of a human subject's skin described herein result in an improvement on the FDA Investigator's Global Assessment (IGA) of Acne Severity compared to baseline. The FDA IGA of Acne Severity is set forth in Table 1:

TABLE 1
FDA IGA of Acne Severity

| Grade | Description |
|---|---|
| 0 | Clear skin with no inflammatory or noninflammatory lesions |
| 1 | Almost clear; rare noninflammatory lesions with |
| | no more than one small inflammatory lesion |
| 2 | Mild severity; greater than Grade 1; some |
| | noninflammatory lesions with no more than a few inflammatory |
| | lesions (papules/pustules only, no nodular lesions) |
| 3 | Moderate severity; greater than Grade 2; up to many |
| | noninflammatory lesions and may have some inflammatory |
| | lesions, but no more than one small nodular lesion |
| 4 | Severe; greater than Grade 3; up to many noninflammatory |
| | and inflammatory lesions, but no more than a few |
| | nodular lesions |

In some embodiments, methods for improving the appearance of a human subject's skin described herein result in a decrease in the number of acne lesions on a human subject's skin compared to baseline. Any suitable area may be used for the counts, for example, the human subject's face. Acne lesions include inflammatory lesions, such as papules, pustules, and cysts/nodules, as well as non-inflammatory lesions, such as open comedones and closed comedones.

In some embodiments, methods for improving the appearance of a human subject's skin described herein result in an improvement in clinical grading of efficacy parameters. Any suitable grading scale known in the art or described herein may be used.

In some embodiments, methods for improving the appearance of a human subject's skin described herein result in an improvement in corneometer measurements compared to baseline measurements. The electrical properties of the skin and its capacitance are largely determined by the water content of the horny layer. The Corneometer is an instrument designed to measure changes in the capacitance of the skin resulting from changes in the degree of hydration. It is particularly sensitive to low hydration levels. The Corneometer expresses the capacitance of the skin surface in arbitrary units of skin hydration.

In some embodiments, methods for improving the appearance of a human subject's skin described herein result in an improvement in tewameter measurements. A tewameter may be used to measure the trans-epidermal water loss (TEWL) from the skin. This is a readout of skin integrity and barrier functions.

In some embodiments, methods for improving the appearance of a human subject's skin described herein result in an improvement in sebumeter measurements compared to baseline sebumeter measurements. The sebumeter is a commercially available instrument designed to evaluate the sebum content of the skin in μg/cm². It photometrically measures the increase in the transparency of a special translucent plastic strip when it becomes coated with sebum. The plastic strip is approximately 0.1 mm thick and 64 mm² in area. It is housed in the portable head of the instrument. The strip is backed by a mirror, which presses it against the relevant skin with a fixed pressure of 10N by means of a spring. The instrument also contains a timing device that limits the time of contact that the strip with the skin to thirty (30) seconds. The transparency of the plastic strip is evaluated by means of a microprocessor and is read off a digital instrument directly as μg of sebum per square centimeter.

### Kits

In some embodiments, kits may contain any combination of the compositions and their ingredients described herein. The kit may further comprise instructions for use.

The invention will be further described by way of illustration in the following example and by reference to the accompanying diagrammatic drawings in which:

### BRIEF DESCRIPTION OF THE DRAWINGS

FIG. 1 is a flow chart illustrating a study design of a 12-week single-arm prospective study described in Example 1.

FIG. 2 is a list of ingredients contained in each capsule administered to participants of the study described in Example 1.

FIG. 3 shows the bioinstrumentation measurements of hydration via corneometer and sebum via sebumeter as described in Example 1 and indicating a decrease in sebum (sebumeter) and increased hydration (corneometer). No significant change detected in transepidermal water loss (TEWL). Measurements taken at baseline, week 4, week 8 and week 12. The p-value indicates 12-week value compared to baseline.

FIG. 4A-B depicts the results by IGA of acne severity, and inflammatory and non-inflammatory lesion counts (FIG. 4A) as well as clinical grading of skin quality parameters (FIG. 4B) which progressively improved during the duration of the study as described in Example 1 and includes the percent of subjects improved at Week 12 compared to baseline in clinically graded skin quality parameters showing a progressive improvement of most parameters throughout the duration of the study. p-value indicates 12-week values analyzed compared to baseline.

FIG. 5 depicts the self-assessment questionnaire results as described in Example 1 showing percent of favorable responses for weeks 4, 8, and 12. Significance determined by comparing proportion of favorable responses to unfavorable responses for each statement. *p<0.05, **p<0.01, ***p<0.001.

FIG. 6 depicts face locations used in the analysis which was performed as described in Example 1.

FIGS. 7A-7F depicts before (top) and after (bottom) images from week 4 to week12 in individuals undergoing intervention described in Example 1.

The example described in this section is provided for illustration only and is not intended to limit the claimed invention.

### EXAMPLE 1

### A Clinical Study to Evaluate the Benefit of a Dietary Supplement on Skin When Consumed by Adults With Facial Acne Conditions

A single-center cosmetic clinical trial was conducted to assess the efficacy of a dietary supplement when taken over the course of 12 weeks by women and men in good general health, having Fitzpatrick skin type I-VI and having facial acne at one of the following severity levels:
(i) Mild to moderate facial acne (score of 2-3 according to the FDA IGA of Acne Severity) with at least 3 inflammatory lesions and at least 5 non-inflammatory lesions on the face at baseline OR
(ii) Severe facial acne (score of 4 according to the FDA IGA of Acne Severity) with at least 5inflammatory lesions and at least 5 non-inflammatory lesions on the face at baseline (at least n=3 and maximum of n=7 subjects).

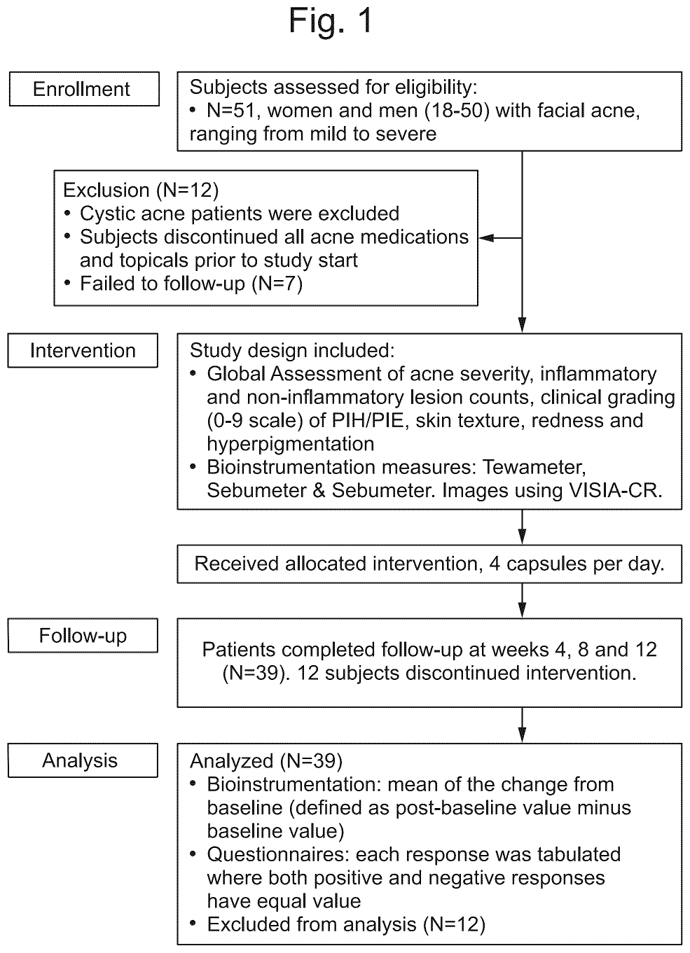

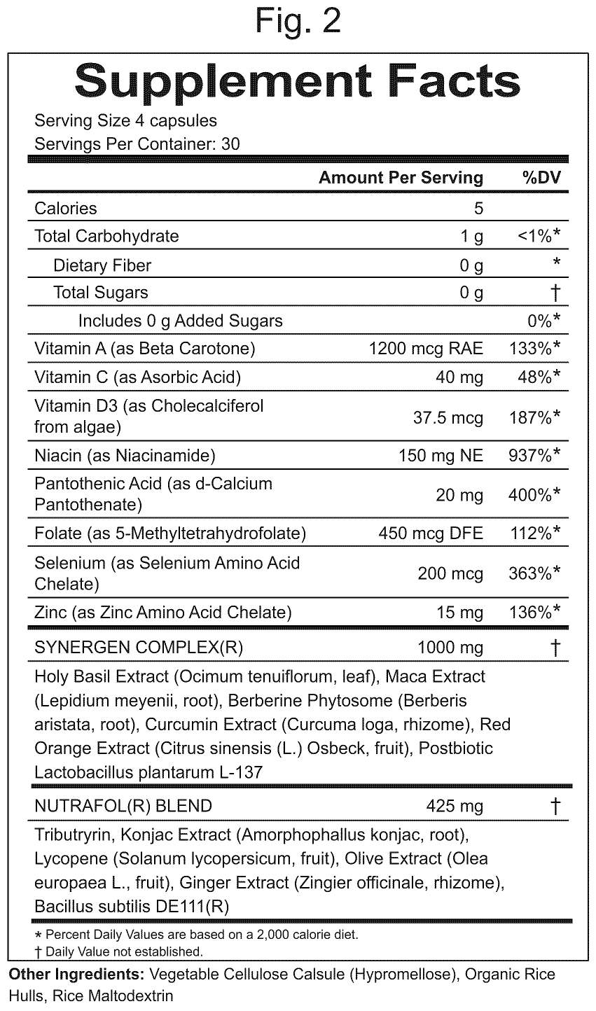

Fifty-one subjects were recruited with 39 subjects (mean age=31.5±7.7 years, range=19-48) completing the study as outlined in FIG. 1, with daily treatment with four capsules, each containing the ingredients listed in FIG. 2. Subject demographics were female (67%), White/Caucasian (41%, 6:10 M: F ratio), Black (41%, 5:11 M: F ratio), and Asian (13%, 1:3 M: F ratio).

### Assessment Protocol

### FDA IGA of Acne Severity

At baseline and weeks 4, 8, and 12, an expert grader evaluates each subject's global face using the FDA IGA scale, with half scores used as necessary.

### Acne Lesion Counts

Acne lesion counts are performed at baseline and weeks 4, 8, and 12. A clinical evaluator counts and records the number of papules, pustules, open comedones, closed comedones, and cysts/nodules on each subject's face. Papules, pustules, and cysts/nodules are classified as inflammatory acne lesions while open and closed comedones are classified as non-inflammatory lesions.

Acne lesion and macule counts are assessed separately for each of the following facial locations/quadrants: forehead, left cheek (including left side of nose), chin (including the area above the upper lip), and right cheek (including right side of nose). Lesions under the jawline or along the hairline (including eyebrows) will not be included in the counts.

### Clinical Grading of Efficacy Parameters

Clinical grading of efficacy parameters is performed at baseline and weeks 4, 8, and 12. The efficacy parameters are assessed by the expert grader at the indicated locations using a modified Griffiths 10-point scale according to the following numerical definitions. Half-point scores may be used as necessary to describe the skin condition more accurately.
0=none (best possible condition)
1 to 3=mild
4 to 6=moderate
7 to 9=severe (worst possible condition)

The following parameters are evaluated using the indicated scale anchors:

### Evaluation Parameters

| Parameter | Location | 0= | 9= |
|---|---|---|---|
| Skin texture | Cheek | Smooth, even-looking | Rough, uneven- |
| (visual) | | skin texture, | looking skin texture |
| | | no roughness | |
| Skin smoothness | Cheek | Smooth, even-feeling | Rough, uneven- |
| (tactile) | | skin texture | feeling skin texture |
| Hyper- | Global | Even skin color, no | Significant (severe) |
| pigmentation | face | hyperpigmentation | hyperpigmented |
| | | | appearance, involving |
| | | | most of the face, with |
| | | | very strong intensity |
| Acne marks post- | Global | Even skin color, no | Pronounced PIH/PIE, |
| inflammatory | face | PIH/PIE present | numerous/extensive |
| hyperpigmentation/ | | | presence of reddish |
| post-inflammatory | | | or brown PIH/PIE |
| erythema | | | lesions |
| (PIH/PIE) | | | |
| Redness | Global | Even, healthy | Pronounced areas of |
| | face | skin tone | redness and deep |
| | | with no redness | intense red color |

### Bioinstrumentation Measurements

Corneometer Measurements: Corneometer measurements are performed at baseline and weeks 4, 8, and 12. Triplicate Corneometer measurements are taken on the center of each subject's right or left cheek (at the intersection of lines extending up from the corner of the mouth and horizontally across the bottom of the nose) according to a predetermined randomization. The Corneometer CM 825 (Courage+Khazaka electronic GmbH, Koln,

Germany), in conjunction with product treatment, is used to measure product hydration effects on the skin surface. The Corneometer measures moisture content in the stratum corneum by an electrical capacitance method. The measurement has no units but is proportional to the dielectric constant of the surface layers of the skin and increases as the skin becomes more hydrated. The readings are directly related to the skin's electrical capacitance (picofarads).

Tewameter Measurements: Tewameter measurements are performed at baseline and weeks 4, 8, and 12. Duplicate Tewameter measurements are taken on the center of each subject's right or left cheek (at the intersection of lines extending up from the corner of the mouth and horizontally across the bottom of the nose) on the opposite side from where Corneometer measurements were taken. The Tewameter TM300 or Tewameter Triple TM330T (Courage+Khazaka electronic GmbH, Koln, Germany) measures the passive transfer of water through the stratum corneum (transepidermal water loss (TEWL)). The measurement of this water evaporation is based on the diffusion principle in an open chamber and the density gradient is measured indirectly by two pairs of sensors located inside the hollow cylindrical probe. Data are analyzed by a microprocessor and reported in g/m2/h. A decrease in TEWL values reflects an improvement in the barrier properties of the skin.

Sebumeter Measurements: Sebumeter measurements are performed at baseline and weeks 4, 8, and 12. Duplicate Sebumeter measurements are taken on non-lesion, adjacent, nonoverlapping areas of each subject's forehead, with the location marked on a facial diagram. The Sebumeter SM 815 (Courage+Khazaka electronic GmbH, Koln, Germany) measures sebum quantity on the skin using a photometric method. This method is independent of moisture. The measurement is the transmission of light through sebutape, a synthetic material that becomes transparent upon contact with sebum. A microprocessor in the Sebumeter calculates the amount (μg sebum/cm²) on the skin surface based on the transparency of the sebutape.

### Digital Imaging Procedures

VISIA-CR Imaging Procedures: VISIA-CR imaging procedures are performed at baseline and weeks 4, 8, and 12. Digital images are taken of each subject's face (left, front, and right views) (3 total) using the VISIA-CR photo station (Canfield Imaging Systems, Fairfield, New Jersey) with a Canon R5 digital SLR camera (Canon Incorporated, Tokyo, Japan) and LED flash illumination under the following lighting conditions:
Standard clinical: visible (bright)
Cross-polarized
Parallel polarized
Blue fluorescence

### Microbiome Sampling Procedures

Subjects participate in microbiome sampling procedures at baseline and week 12. Each subject will have 2 microbiome sampling test sites: 1 on the forehead and 1 on the left or right cheek, as determined by the grader or designee. Facial diagrams for each subject are marked for swab sites at baseline to ensure the samples are taken from the same locations at all time points. Surface skin microflora samples are collected from each subject's swab test site (approximately 4 cm² area of the skin) using a non-invasive swab technique.

### Self-Assessment Questionnaires

Subjects complete a self-assessment questionnaire at baseline and weeks 4, 8, and 12.

### Results

Analysis of Self-Assessment Questionnaires. Scores range from 1-4, where 1 is most favorable and 4 is most unfavorable.

| | | | | |
|---|---|---|---|---|
| | | Binomial Test | Favorable (1, 2) | Unfavorable (3, 4) |
| Time Point | N | P Value* | N(%) | N(%) |

My skin feels nourished.

| | | | | |
|---|---|---|---|---|
| Week 4 | 38 | <.001 | 35(92.1) | 3(7.9) |
| Week 8 | 39 | <.001 | 36(92.3) | 3(7.7) |
| Week 12 | 39 | <.001 | 36(92.3) | 3(7.7) |

My skin feels more hydrated.

| | | | | |
|---|---|---|---|---|
| Week 4 | 38 | <.001 | 30(78.9) | 8(21.1) |
| Week 8 | 39 | <.001 | 35(89.7) | 4(10.3) |
| Week 12 | 39 | <.001 | 32(82.1) | 7(17.9) |

My skin tone looks more even.

| | | | | |
|---|---|---|---|---|
| Week 4 | 38 | 0.034 | 26(68.4) | 12(31.6) |
| Week 8 | 39 | 0.003 | 29(74.4) | 10(25.6) |
| Week 12 | 39 | <.001 | 33(84.6) | 6(15.4) |

My skin has a brighter complexion.

| | | | | |
|---|---|---|---|---|
| Week 4 | 38 | 0.034 | 26(68.4) | 12(31.6) |
| Week 8 | 39 | <.001 | 31(79.5) | 8(20.5) |
| Week 12 | 39 | <.001 | 34(87.2) | 5(12.8) |

My skin feels softer.

| | | | | |
|---|---|---|---|---|
| Week 4 | 38 | 0.073 | 25(65.8) | 13(34.2) |
| Week 8 | 39 | <.001 | 32(82.1) | 7(17.9) |
| Week 12 | 39 | <.001 | 33(84.6) | 6(15.4) |

My skin texture feels smoother.

| | | | | |
|---|---|---|---|---|
| Week 4 | 38 | 0.034 | 26(68.4) | 12(31.6) |
| Week 8 | 39 | 0.003 | 29(74.4) | 10(25.6) |
| Week 12 | 39 | <.001 | 34(87.2) | 5(12.8) |

My skin is less oily.

| | | | | |
|---|---|---|---|---|
| Week 4 | 38 | 0.005 | 28(73.7) | 10(26.3) |
| Week 8 | 39 | 0.003 | 29(74.4) | 10(25.6) |
| Week 12 | 39 | 0.003 | 29(74.4) | 10(25.6) |

My skin imperfections have improved.

| | | | | |
|---|---|---|---|---|
| Week 4 | 38 | 0.034 | 26(68.4) | 12(31.6) |
| Week 8 | 39 | <.001 | 31(79.5) | 8(20.5) |
| Week 12 | 39 | <.001 | 32(82.1) | 7(17.9) |

I have less blackheads.

| | | | | |
|---|---|---|---|---|
| Week 4 | 38 | 0.418 | 22(57.9) | 16(42.1) |
| Week 8 | 39 | 0.053 | 26(66.7) | 13(33.3) |
| Week 12 | 39 | 0.053 | 26(66.7) | 13(33.3) |

My pores are less noticeable.

| | | | | |
|---|---|---|---|---|
| Week 4 | 38 | 0.143 | 24(63.2) | 14(36.8) |
| Week 8 | 39 | 0.003 | 29(74.4) | 10(25.6) |
| Week 12 | 39 | 0.108 | 25(64.1) | 14(35.9) |

I have less breakouts.

| | | | | |
|---|---|---|---|---|
| Week 4 | 38 | 0.256 | 23(60.5) | 15(39.5) |
| Week 8 | 39 | <.001 | 32(82.1) | 7(17.9) |
| Week 12 | 39 | 0.001 | 30(76.9) | 9(23.1) |

My skin is clearer.

| | | | | |
|---|---|---|---|---|
| Week 4 | 38 | 0.143 | 24(63.2) | 14(36.8) |
| Week 8 | 39 | <.001 | 32(82.1) | 7(17.9) |
| Week 12 | 39 | <.001 | 34(87.2) | 5(12.8) |

My acne has improved.

| | | | | |
|---|---|---|---|---|
| Week 4 | 38 | 0.014 | 27(71.1) | 11(28.9) |
| Week 8 | 39 | <.001 | 34(87.2) | 5(12.8) |
| Week 12 | 39 | <.001 | 34(87.2) | 5(12.8) |

My skin is clear of acne.

| | | | | |
|---|---|---|---|---|
| Week 4 | 38 | 0.005 | 10(26.3) | 28(73.7) |
| Week 8 | 39 | 0.053 | 13(33.3) | 26(66.7) |
| Week 12 | 39 | 0.200 | 15(38.5) | 24(61.5) |

I’d recommend this product to a friend.

| | | | | |
|---|---|---|---|---|
| Week 12 | 39 | <.001 | 36(92.3) | 3(7.7) |

I would add this product to my skin care routine.

| | | | | |
|---|---|---|---|---|
| Week 12 | 39 | <.001 | 33(84.6) | 6(15.4) |

I would replace an existing product, with this one, in my skin care routine.

| | | | | |
|---|---|---|---|---|
| Week 12 | 39 | 0.108 | 25(64.1) | 14(35.9) |

My skin is less red.

| | | | | |
|---|---|---|---|---|
| Week 4 | 28 | 0.185 | 18(64.3) | 10(35.7) |
| Week 8 | 34 | <.001 | 29(85.3) | 5(14.7) |
| Week 12 | 33 | <.001 | 28(84.8) | 5(15.2) |

My skin is less irritated.

| | | | | |
|---|---|---|---|---|
| Week 4 | 34 | 0.121 | 22(64.7) | 12(35.3) |
| Week 8 | 36 | <.001 | 29(80.6) | 7(19.4) |
| Week 12 | 37 | <.001 | 30(81.1) | 7(18.9) |

I have less dark spots.

| | | | | |
|---|---|---|---|---|
| Week 4 | 37 | 1.000 | 19(51.4) | 18(48.6) |
| Week 8 | 37 | 0.099 | 24(64.9) | 13(35.1) |
| Week 12 | 37 | 0.008 | 27(73.0) | 10(27.0) |

My skin is less sensitive.

| | | | | |
|---|---|---|---|---|
| Week 4 | 33 | 0.296 | 20(60.6) | 13(39.4) |
| Week 8 | 35 | 0.090 | 23(65.7) | 12(34.3) |
| Week 12 | 37 | 0.099 | 24(64.9) | 13(35.1) |

My skin health has improved.

| | | | | |
|---|---|---|---|---|
| Week 4 | 37 | 0.003 | 28(75.7) | 9(24.3) |
| Week 8 | 39 | <.001 | 34(87.2) | 5(12.8) |
| Week 12 | 37 | <.001 | 34(91.9) | 3(8.1) |

My chest acne has improved.

| | | | | |
|---|---|---|---|---|
| Week 4 | 22 | 0.134 | 15(68.2) | 7(31.8) |
| Week 8 | 23 | 0.405 | 14(60.9) | 9(39.1) |
| Week 12 | 22 | 0.134 | 15(68.2) | 7(31.8) |

My back acne has improved.

| | | | | |
|---|---|---|---|---|
| Week 4 | 21 | 0.664 | 12(57.1) | 9(42.9) |
| Week 8 | 22 | 0.286 | 14(63.6) | 8(36.4) |
| Week 12 | 21 | 0.078 | 15(71.4) | 6(28.6) |

I feel less stressed.

| | | | | |
|---|---|---|---|---|
| Week 4 | 35 | 1.000 | 18(51.4) | 17(48.6) |
| Week 8 | 33 | 0.487 | 19(57.6) | 14(42.4) |
| Week 12 | 34 | 0.058 | 23(67.6) | 11(32.4) |

I am sleeping better.

| | | | | |
|---|---|---|---|---|
| Week 4 | 35 | 0.500 | 20(57.1) | 15(42.9) |
| Week 8 | 34 | 0.392 | 20(58.8) | 14(41.2) |
| Week 12 | 32 | 0.597 | 18(56.3) | 14(43.8) |

My confidence has improved.

| | | | | |
|---|---|---|---|---|
| Week 4 | 35 | 0.311 | 21(60.0) | 14(40.0) |
| Week 8 | 37 | <.001 | 30(81.1) | 7(18.9) |
| Week 12 | 34 | <.001 | 30(88.2) | 4(11.8) |

*Calculated from binomial test (sign test). The null hypothesis is that the proportion of the favorable response is equal to the unfavorable response. Subjects with N/A responses have been excluded from the analysis.

### Change from Baseline Statistics for IGA of Acne Severity

| | | | | | | | | |
|---|---|---|---|---|---|---|---|---|
| | Time | | Subject | Subject | Mean | SD for | Mean | Signed Rank |
| Detail | Point | N | Improved, % | Worsened, % | Change | Change | Change % | Test P Value** |

FDA Investigator's Global Assessment (IGA) of Acne severity

| | | | | | | | | |
|---|---|---|---|---|---|---|---|---|
| Global | Week 4 | 38 | 60.5 | 2.6 | −0.36 | 0.38 | −14.3 | <.001 |
| face | Week 8 | 39 | 76.9 | 7.7 | −0.55 | 0.54 | −22.3 | <.001 |
| | Week 12 | 39 | 84.6 | 0.0 | −0.74 | 0.52 | −30.1 | <.001 |

**Calculated from Wilcoxon signed rank test. The null hypothesis is that the mean change from baseline is zero.

### Change from Baseline Statistics for Acne Lesion Counts

| | | | | | | | | |
|---|---|---|---|---|---|---|---|---|
| | Time | | Subject | Subject | Mean | SD for | Mean | Signed Rank |
| Detail | Point | N | Improved, % | Worsened, % | Change | Change | Change % | Test P Value** |

Total Inflammatory

| | | | | | | | | |
|---|---|---|---|---|---|---|---|---|
| Global | Week 4 | 38 | 78.9 | 18.4 | −3.24 | 4.55 | −40.2 | <.001 |
| face | Week 8 | 39 | 82.1 | 15.4 | −3.69 | 5.26 | −46.5 | <.001 |
| | Week 12 | 39 | 69.2 | 28.2 | −2.79 | 5.51 | −35.2 | <.001 |

Total Non-inflammatory

| | | | | | | | | |
|---|---|---|---|---|---|---|---|---|
| Global | Week 4 | 38 | 76.3 | 21.1 | −4.42 | 7.86 | −22.4 | <.001 |
| face | Week 8 | 39 | 84.6 | 15.4 | −6.00 | 7.41 | −31.0 | <.001 |
| | Week 12 | 39 | 87.2 | 12.8 | −9.05 | 11.07 | −46.7 | <.001 |

**Calculated from Wilcoxon signed rank test. The null hypothesis is that the mean change from baseline is zero.

### Change from Baseline Statistics for Clinical Grading of Efficacy Parameters

| | | | | | | | | |
|---|---|---|---|---|---|---|---|---|
| | Time | | Subject | Subject | Mean | SD for | Mean | Signed Rank |
| Detail | Point | N | Improved, % | Worsened, % | Change | Change | Change % | Test P Value** |

Skin texture (visual)

| | | | | | | | | |
|---|---|---|---|---|---|---|---|---|
| Cheek | Week 4 | 38 | 60.5 | 5.3 | −0.32 | 0.36 | −6.3 | <.001 |
| | Week 8 | 39 | 69.2 | 0.0 | −0.41 | 0.32 | −8.3 | <.001 |
| | Week 12 | 39 | 84.6 | 2.6 | −0.58 | 0.39 | −11.6 | <.001 |

Skin smoothness (tactile)

| | | | | | | | | |
|---|---|---|---|---|---|---|---|---|
| Cheek | Week 4 | 38 | 63.2 | 10.5 | −0.32 | 0.46 | −7.3 | <.001 |
| | Week 8 | 39 | 69.2 | 10.3 | −0.36 | 0.51 | −8.2 | <.001 |
| | Week 12 | 39 | 84.6 | 5.1 | −0.51 | 0.49 | −11.8 | <.001 |

Hyperpigmentation

| | | | | | | | | |
|---|---|---|---|---|---|---|---|---|
| Global | Week 4 | 38 | 28.9 | 2.6 | −0.14 | 0.28 | −3.3 | 0.006 |
| face | Week 8 | 39 | 43.6 | 2.6 | −0.23 | 0.34 | −5.2 | <.001 |
| | Week 12 | 39 | 66.7 | 0.0 | −0.36 | 0.30 | −8.2 | <.001 |

Acne marks (PIH/PIE)

| | | | | | | | | |
|---|---|---|---|---|---|---|---|---|
| Global | Week 4 | 38 | 42.1 | 5.3 | −0.28 | 0.49 | −5.8 | <.001 |
| face | Week 8 | 39 | 64.1 | 7.7 | −0.40 | 0.48 | −8.4 | <.001 |
| | Week 12 | 39 | 79.5 | 5.1 | −0.56 | 0.50 | −11.9 | <.001 |

Redness

| | | | | | | | | |
|---|---|---|---|---|---|---|---|---|
| Global | Week 4 | 32 | 56.3 | 3.1 | −0.44 | 0.54 | −15.2 | <.001 |
| face | Week 8 | 33 | 69.7 | 15.2 | −0.48 | 0.64 | −16.7 | <.001 |
| | Week 12 | 33 | 78.8 | 9.1 | −0.55 | 0.64 | −18.8 | <.001 |

**Calculated from Wilcoxon signed rank test. The null hypothesis is that the mean change from baseline is zero. Subjects with a baseline value 0 for any parameter have been excluded from analysis for that specific parameter.

### Change from Baseline Statistics for Corneometer Measurements

| | | | | | | | | |
|---|---|---|---|---|---|---|---|---|
| | Time | | Subject | Subject | Mean | SD for | Mean | Paired T Test |
| Detail | Point | N | Improved, % | Worsened, % | Change | Change | Change % | P Value*** |

Corneometer Measurements

| | | | | | | | | |
|---|---|---|---|---|---|---|---|---|
| Cheek | Week 4 | 38 | 68.4 | 31.6 | 6.59 | 12.11 | 18.9 | 0.002 |
| | Week 8 | 39 | 71.8 | 28.2 | 13.86 | 17.28 | 39.5 | <.001 |
| | Week 12 | 39 | 74.4 | 25.6 | 7.70 | 14.51 | 22.0 | 0.002 |

***Calculated from paired t test. The null hypothesis is that the mean change from baseline is zero.

### Change from Baseline Statistics for Tewameter Measurements

| | | | | | | | | |
|---|---|---|---|---|---|---|---|---|
| | Time | | Subject | Subject | Mean | SD for | Mean | Paired T Test |
| Detail | Point | N | Improved, % | Worsened, % | Change | Change | Change % | P Value*** |

Tewameter Measurements

| | | | | | | | | |
|---|---|---|---|---|---|---|---|---|
| Cheek | Week 4 | 38 | 47.4 | 50.0 | −0.08 | 5.46 | −0.4 | 0.927 |
| | Week 8 | 39 | 41.0 | 59.0 | −0.03 | 7.18 | −0.2 | 0.976 |
| | Week 12 | 39 | 41.0 | 59.0 | 1.34 | 9.03 | 7.2 | 0.361 |

***Calculated from paired t test. The null hypothesis is that the mean change from baseline is zero.

### Change from Baseline Statistics for Sebumeter Measurements

| | | | | | | | | |
|---|---|---|---|---|---|---|---|---|
| | Time | | Subject | Subject | Mean | SD for | Mean | Paired T Test |
| Detail | Point | N | Improved, % | Worsened, % | Change | Change | Change % | P Value*** |

Sebumeter Measurements

| | | | | | | | | |
|---|---|---|---|---|---|---|---|---|
| Forehead | Week 4 | 38 | 76.3 | 21.1 | −39.68 | 53.42 | −25.3 | <.001 |
| (non-lesion | Week 8 | 38 | 78.9 | 18.4 | −49.46 | 67.62 | −31.6 | <.001 |
| area) | Week 12 | 39 | 71.8 | 28.2 | −36.49 | 68.67 | −23.1 | 0.002 |

***Calculated from paired t test. The null hypothesis is that the mean change from baseline is zero.

### Conclusions

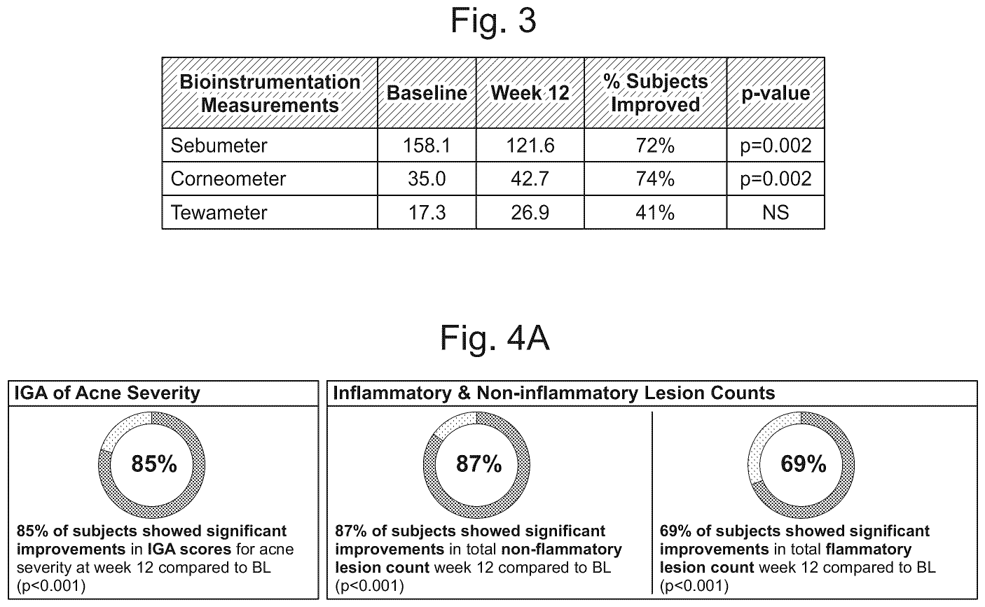

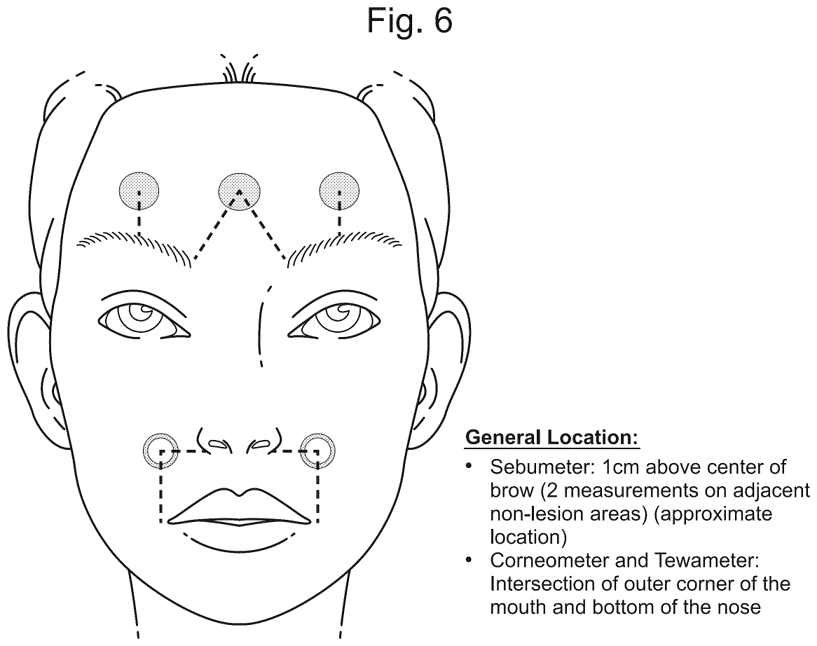

Hydration and sebumeter measurements using the template of FIG. 6 improved significantly compared to baseline over the 12-week treatment period (FIG. 3).

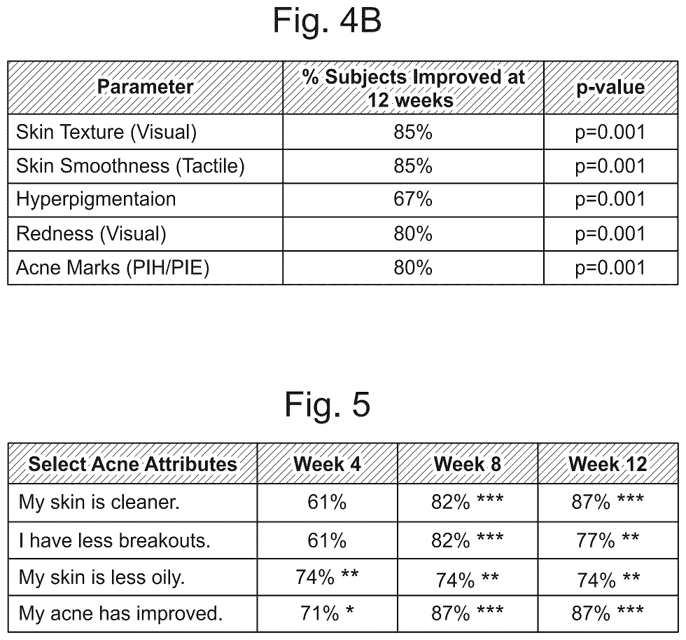

87% of participants rated their skin clearer and 67%-85% of participants rated their skin quality parameters improving during the duration of the study (FIGS. 4A, 4B and 5).

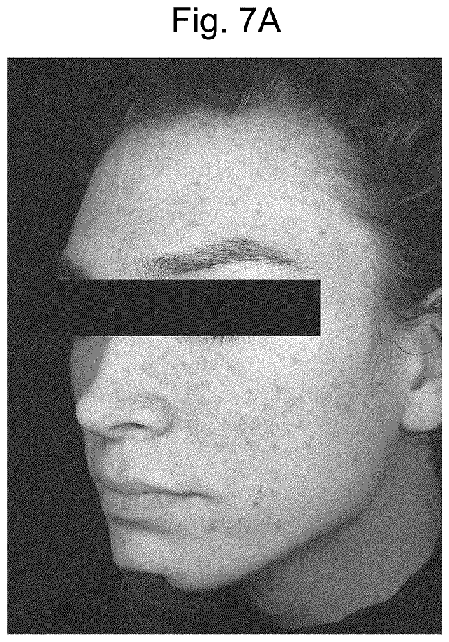

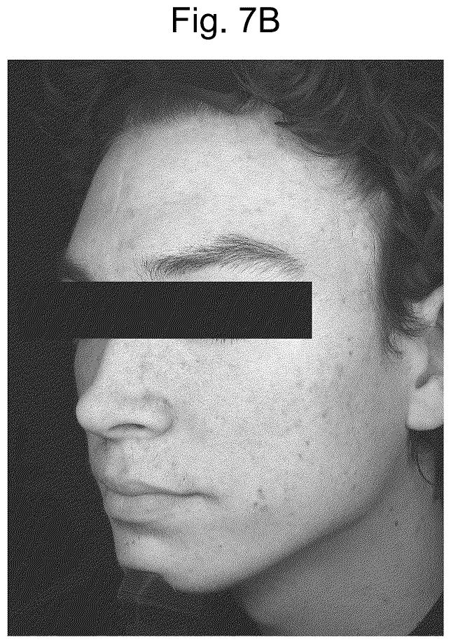

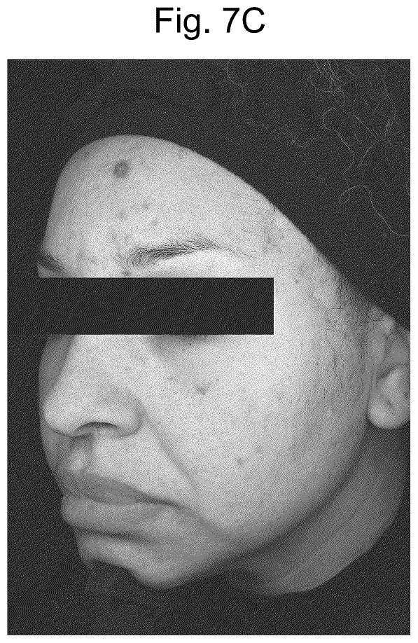

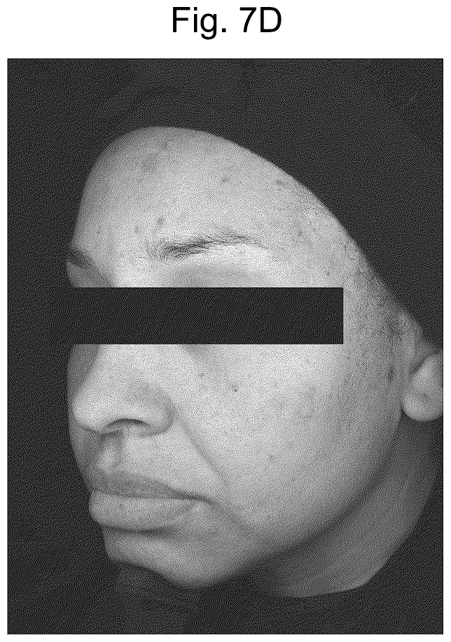

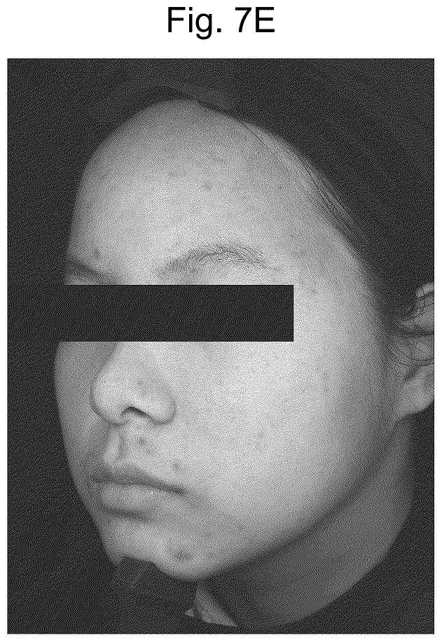

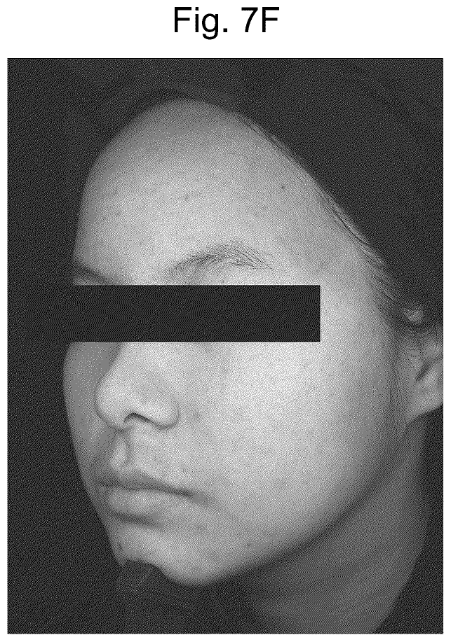

The results show improvements in acne and skin parameters in adults over the course of 12 weeks (FIGS. 7A-7F).

1. A composition comprising:
about 1.0% w/w-about 20% w/w of Lepidium meyenii or an extract thereof;
about 1.0% w/w-about 20% w/w of Berberis aristata, an extract thereof or berberine;
about 1.0% w/w-about 25% w/w of Ociimum tenuiflorum or an extract thereof;
about 1.0% w/w-about 20% w/w of Curcuma longa, an extract thereof, or one or more curcuminoids;
about 0.1% w/w-about 10% of Citrus sinensis (L.) Osbeck or an extract thereof;
about 0.1% w/w-about 10% of inactivated Lactobacillus plantarum;
about 0.0001% w/w-about 5.0% w/w of cholecalciferol;
about 0.1% w/w-about 8% w/w of a pharmaceutically acceptable zinc salt or chelate; and
about 0.1% w/w-about 5.0% w/w of a pharmaceutically acceptable selenium salt or chelate.

2. The composition of claim 1, wherein the composition comprises:
about 25 mg-about 750 mg of Lepidium meyenii or an extract thereof;
about 12.5 mg-about 750 mg of Berberis aristata, an extract thereof or berberine;
about 25 mg-about 500 mg of Ociimum tenuiflorum or an extract thereof;
about 12.5 mg-about 500 mg of Curcuma longa, an extract thereof or one or more curcuminoids;
about 1.25 mg-about 250 mg of Citrus sinensis (L.) Osbeck or an extract thereof;
about 0.25 mg-about 250 mg of inactivated Lactobacillus plantarum;
about 1.25 mcg-about 50 mcg of cholecalciferol;
an amount of a pharmaceutically acceptable zinc salt or chelate that is equivalent to about 0.25 mg-about 12.5 mg of elemental zinc; and
an amount of a pharmaceutically acceptable selenium salt or chelate that is equivalent to about 1.25 mcg-about 125 mcg of elemental selenium.

3. The composition of claim 1, further comprising about 0.5% w/w-about 10% w/w of Olea europaea or an extract thereof.

4. The composition of claim 1, further comprising about 1% w/w-about 15% w/w of Amorphophallus konjac or an extract thereof.

5. The composition of claim 1, further comprising about 1% w/w-about 10% w/w of Solanum lycopersicum or an extract thereof.

6. The composition of claim 1, further comprising about 0.125% w/w-about 5% w/w of Zingiber officinale or an extract thereof.

7. The composition of claim 1, further comprising about 1% w/w-about 20% w/w of tributyrin.

8. The composition of claim 1, further comprising about 0.1% w/w-about 5% w/w of Bacillus subtilis DE111.

9. The composition of claim 1, further comprising about 0.01% w/w-about 5% w/w of beta-carotene.

10. The composition of claim 1, comprising about 0.25% w/w-about 10% w/w of vitamin C.

11. The composition of claim 1, further comprising about 1.0% w/w-about 20.0% w/w of vitamin B3.

12. The composition of claim 1, comprising about 0.1% w/w-about 10% w/w of vitamin B5 or an amount of a pharmaceutically acceptable salt thereof that is equivalent to about 0.1% w/w-about 10% w/w of vitamin B5.

13. The composition of claim 1, further comprising about 0.005% w/w-about 1% w/w of L-5-methyltetrahydrofolate or an amount of a pharmaceutically acceptable salt thereof that is equivalent to about 0.005% w/w-about 1% w/w of L-5-methyltetrahydrofolate.

14. A method for improving the appearance of skin of a human subject in need of skin appearance improvement, preferably wherein the human subject has acne, comprising administering to the human subject an effective amount of the composition according to claim 1.
